(19) United States
(12) Patent Application Publication
(10) Pub. No.: US 2017/0347691 A1
(43) Pub. Date: Dec. 7, 2017

(54) METHOD FOR MANUFACTURING SMILAX CHINA L. ROOT EXTRACT HAVING INCREASED ACTIVE INGREDIENTS AND BEVERAGE COMPOSITION CONTAINING THE EXTRACT FOR DETOXIFICATION

(71) Applicant: INDUSTRY-ACADEMIA COOPERATION GROUP OF SEJONG UNIVERSITY, Seoul (KR)

(72) Inventors: Soon Mi SHIM, Yongin-si (KR); Tae Sik PARK, Seongnam-si (KR); Jae Hwan CHUNG, Seoul (KR); Kyung Moo KIM, Seoul (KR); So Ra YOON, Incheon (KR); Ui Jeong YANG, Guri-si (KR); Yu Ra SON, Seoul (KR); Joo Won SUH, Yongin-si (KR); Seung Hwan YANG, Yongin-si (KR)

(73) Assignee: INDUSTRY-ACADEMIA COOPERATION GROUP OF SEJONG UNIVERSITY, Seoul (KR)

(21) Appl. No.: 15/646,803

(22) Filed: Jul. 11, 2017

Related U.S. Application Data
(63) Continuation of application No. 14/305,795, filed on Jun. 16, 2014, now abandoned.

(30) Foreign Application Priority Data
Jun. 17, 2013 (KR) 10-2013-0068836
Jun. 17, 2013 (KR) 10-2013-0068837
May 27, 2014 (KR) 10-2014-0063706
Jun. 13, 2014 (KR) 10-2014-0072126

(51) Int. Cl. A23L 2/52; A61K 31/7034; A61K 36/90; A61K 31/05; A23L 33/105; A61K 9/00

(52) U.S. Cl. CPC A23L 2/52; A61K 31/05; A23L 33/105; A61K 31/7034; A61K 36/90; A23V 2002/00; A23V 2250/21; A61K 9/0095; A61K 2236/19

(57) ABSTRACT

A method for preparing a Smilax china L. root extract having an increased active ingredient content, and a beverage composition for detoxification including the same are provided. The method for preparing a Smilax china L. root extract includes preparing a powder of a Smilax china L. root, obtaining an ethanol extract of Smilax china L. root from the powder of Smilax china L. root using ethanol, and concentrating the ethanol extract of Smilax china L. root under a reduced vacuum to obtain a concentrate having a reduced ethanol content.

### CROSS-REFERENCE TO RELATED APPLICATION

Any and all applications for which a foreign or domestic priority claim is identified in the Application Data Sheet as filed with the present application are hereby incorporated by reference under 37 CFR 1.57.

### BACKGROUND

### 1. Field

The present disclosure relates to a plant extract and a beverage composition including the extract, and more particularly, to a method for preparing and/or using a Smilax china L. root extract, and a beverage composition for detoxification including the extract.

### 2. Discussion of Related Art

Cigarette smoking becomes the cause of various diseases, and approximately 4,000 chemical components are produced during cigarette smoking, and inhaled into the human body. In recent years, the smoking population has increased. In particular, smokers are younger and younger, and the number of female smokers tends to increase rapidly. Accordingly, the smoking-related damage cases have been frequently reported. Among these, the main causative materials are nicotine and metabolites thereof which are inhaled by active/passive smoking. These materials cause cancerous diseases in various organs, such as upper gastrointestinal cancer, urinary tract cancer, pancreatic cancer, and the like. Also, these materials have further negative effects which causes pulmonary emphysema by degrading the pulmonary tissue's innate elasticity. Further, nicotine has been recognized as a major chemical that causes heart diseases. In addition, an increase in blood cholesterol and a decrease in vitamins E and C caused by the weakened antioxidative system due to free radicals produced during cigarette smoking have been reported.

Meanwhile, the alcohol consumption in Korea is ranked fourth in the world, and thus the excessive alcohol intake has been an issue. In general, alcohol dehydrogenase (ADH) converts alcohols absorbed into the human body into acetaldehydes in the cytoplasm, and aldehyde dehydrogenase (ALDH) then converts the acetaldehydes into acetyl-CoA, which is then converted into acetates. A large amount of acetaldehydes, which is a hepatotoxic material causing damage to the liver, is produced upon the excessive alcohol intake, resulting in liver damage. In this case, an increase in NADH derived from the alcohol metabolism accelerates oxygen consumption in the liver, thereby causing damage to hepatic tissues.

Meanwhile, Smilax china L. is a climbing deciduous shrub of Liliaceae growing dominantly in the entire mountains and fields in Korea. The root of Smilax china L. is a natural plant resource having various health functionalities, but Smilax china L. may cause damage to other trees and shrubs by hindering photosynthesis and movement of nutriment and moisture in the trees and shrubs while the stem of the plant winds up the surrounding trees and crops, and thus is designated as a plant to be removed for afforestation. Meanwhile, the root of Smilax china L. has been used as a medicinal herb, but its applications are limited and there are scarce scientific studies on its medicinal effects.

The disclosure of this section is to provide background of the invention. Applicant notes that this section may contain information available before this application. However, by providing this section, Applicant does not admit that any information contained in this section constitutes prior art.

### SUMMARY

One aspect of the present invention is directed to developing higher value-added food materials using active ingredients of a root of Smilax china L. which is a plant that is repellent to grow since the plant may cause damage to other trees and shrubs.

However, the technical aspects of the present invention are not limited thereto, and other aspects of the present invention which are not disclosed herein will become more apparent to those of ordinary skill in the art by describing in detail embodiments thereof.

According to an aspect of the present invention, there is provided a method for preparing a Smilax china L. root extract. First, a powder of Smilax china L. root is prepared. An ethanol extract of Smilax china L. root is obtained from the powder of Smilax china L. root using ethanol. The ethanol extract of Smilax china L. root is concentrated under a reduced vacuum to obtain a concentrate having a reduced ethanol content.

In this case, the obtaining of the ethanol extract of Smilax china L. root may include mixing an aqueous solution of 40% to 60% ethanol with the powder of Smilax china L. root, and stirring the resulting mixture at a temperature of 40 to 60° C. at a stirring rate of 50 to 100 rpm. Here, the ethanol extract of Smilax china L. root may be concentrated at a temperature of 40 to 80° C. under a reduced vacuum.

According to another embodiment, the obtaining of the ethanol extract of Smilax china L. root may include mixing the powder of Smilax china L. root, an ethanol material powder, a malt, a yeast, and water to obtain a mixture, and fermenting the mixture to obtain an ethanol extract of Smilax china L. root.

The ethanol material powder may be at least one kind of grain flour selected from the group consisting of rice flour, wheat flour, barley flour, corn flour, sorghum flour, and millet flour. The malt may be at least one kind of malt selected from the group consisting of Aspergillus qwamori, Aspergillus saitoi, Aspergillus usami, and Aspergillus oryzae. The yeast may be at least one kind of yeast selected from the group consisting of Brettanomyces, Candida, Kloeckera, Saccharomycodes, Zygosaccharomyces, Aureobasidium, and Ra parisienne.

The mixture may include the powder of Smilax china L. root at 1 to 10 parts by weight, the malt at 0.5 to 1.5 parts by weight, the yeast at 0.1 to 1 part by weight, and the water at 100 to 300 parts by weight, based on 100 parts by weight of the ethanol material powder.

The fermentation may be carried out at room temperature until the alcohol content reaches approximately 15 to 30%. The ethanol extract of Smilax china L. root may include oxyresveratrol, resveratrol, and piceid as active ingredients.

According to another aspect of the present invention, there is provided a beverage composition for detoxification including the Smilax china L. root extract. Here, the beverage composition may include 0.1 to 0.25% (w/v) of the extract of Smilax china L. root, 0.01 to 0.15% (w/v) of a weak acid, 5.5 to 8% (w/v) of a sugar alcohol, 1 to 2.6% (w/v) of a flavoring agent, and the balance of water.

In this case, the weak acid may include at least one selected from the group consisting of citric acid, malic acid, tartaric acid, acetic acid, oxalic acid, tannic acid, butyric acid, lactic acid, glacial acetic acid, and ascorbic acid. The sugar alcohol acid may include at least one selected from the group consisting of xylitol, isomalt, maltitol, sorbitol, erythritol, mannitol, lactitol, and manatol. The flavoring agent acid may include at least one selected from the group consisting of a fruit extract and a herb extract.

The content of the extract of Smilax china L. root may be in a range of 0.15 to 0.21% (w/v), the content of the weak acid may be in a range of 0.05 to 0.13% (w/v), the content of the sugar alcohol may be in a range of 5.8 to 7.8% (w/v), and the content of the flavoring agent may be in a range of 1.4 to 2.3% (w/v). Further, the content of the extract of Smilax china L. root may be in a range of 0.16 to 0.2% (w/v), the content of the weak acid may be in a range of 0.07 to 0.09% (w/v), the content of the sugar alcohol may be in a range of 6.3 to 6.8% (w/v), and the content of the flavoring agent may be in a range of 1.6 to 1.9% (w/v).

The extract of Smilax china L. root may be prepared using the method for preparing a Smilax china L. root extract. Also, the beverage composition for detoxification may be a beverage composition for detoxification that detoxifies hepatotoxicity caused by nicotine or alcohol.

### BRIEF DESCRIPTION OF THE DRAWINGS

The above and other aspects, features, and advantages of the present invention will become more apparent to those of ordinary skill in the art by describing in detail embodiments thereof with reference to the accompanying drawings, in which:

FIG. 1 is a flowchart illustrating a method for preparing a Smilax china L. root extract according to one embodiment of the present invention;

FIG. 2 is a flowchart illustrating a method for preparing a Smilax china L. root extract according to another embodiment of the present invention;

FIG. 3 is a graph showing the results of separating and identifying functional materials of the Smilax china L. root extract according to Evaluation Example 1;

FIG. 4 is a graph showing the cell viabilities in the experimental groups and the control according to Evaluation Example 2;

FIG. 5 is a graph showing the relative DCF fluorescence intensities in the experimental groups and the control according to Evaluation Example 3;

FIG. 6 is a graph obtained by plotting the cotinine contents against incubation times in the experimental groups and the control according to Evaluation Example 4;

FIG. 7 is a graph illustrating the expression levels of a CYP2A6 enzyme in the experimental groups and the control according to Evaluation Example 5;

FIG. 8 is a graph illustrating the absorbance according to the concentrations in the experimental groups and the control according to Evaluation Example 6;

FIG. 9 is a graph showing the relative DCF fluorescence intensities in the experimental groups and the control according to Evaluation Example 7;

FIG. 10 is a graph showing the sensory degrees of beverages according to Preparation Example 1 of beverage composition including a Smilax china L. root extract;

FIG. 11 is a graph showing the results of separating and identifying functional materials of the Smilax china L. root extract according to Evaluation Example 8;

FIG. 12 is a graph showing the results of separating and identifying functional materials of the Smilax china L. root extract according to Evaluation Example 9;

FIGS. 13 and 14 are graphs plotted by analyzing changes in contents of resveratrol and oxyresveratrol in fractions collected from the extracts of Preparation Examples 2 and 3 and Comparative Example on Days 1, 3, 5, 7, and 9; and

FIG. 15 is a graph plotted by analyzing the antioxidant capacities in the fractions collected from the extracts of Preparation Examples 2 and 3 and Comparative Example on Days 1, 3, 5, 7, and 9.

### DETAILED DESCRIPTION OF EMBODIMENTS

Hereinafter, embodiments of the present invention will be described in detail below with reference to the accompanying drawings. While the present invention is shown and described in connection with embodiments thereof, it will be apparent to those skilled in the art that various modifications can be made without departing from the scope of the invention.

Unless specifically stated otherwise, all the technical and scientific terms used in this specification have the same meanings as what are generally understood by a person skilled in the related art to which the present invention belongs. In general, the nomenclatures used in this specification and the experimental methods described below are widely known and generally used in the related art.

Throughout this specification, the term “active ingredient” or “functional material” used herein refers to a bioactive substance that can control physiological functions, especially, an ingredient that can improve an in vivo detoxification effect or reduce the risk of liver damage. In this specification, an extract or a beverage composition may include additives in addition to the active ingredients.

In this specification, the term “Smilax china L. root extract” may refer to an extract obtained by extracting a Smilax china L. root using ethanol. In this case, the Smilax china L. root extract may be any one of a crude extract, a concentrate having a reduced ethanol content obtained by concentrating the crude extract, a diluted solution of the concentrate, and a solid preparation obtained by processing the concentrate.

Method for Preparing a Smilax China L. Root Extract

FIG. 1 is a flowchart illustrating a method for preparing a Smilax china L. root extract according to one embodiment of the present invention.

Referring to FIG. 1, a powder of Smilax china L. root is prepared (S10). Smilax china L. is a climbing deciduous shrub of Liliaceae growing dominantly in the mountains and fields in Korea. A root may be collected from Smilax china L., washed, dried, and ground to prepare a powder of Smilax china L. root. The washing may be carried out using distilled water, and the drying may be carried out using an air drying or freeze-drying method. Also, the powder of Smilax china L. root may be ground in the form of an extra-fine powder having a mesh size of approximately 400 to approximately 600, and stored below zero temperatures.

Next, an ethanol extract of Smilax china L. root is obtained from the powder of Smilax china L. root using ethanol (S20). More particularly, an aqueous solution of ethanol is mixed with the powder of Smilax china L. root, and the resulting mixture is stirred to obtain an ethanol extract of Smilax china L. root. The aqueous solution of ethanol may have an approximately medium ethanol content, for example, an ethanol content of approximately 40% to approximately 60%, particularly, an ethanol content of approximately 45 to 55%. Also, the stirring may be carried out at a middle temperature, for example, a temperature of approximately 40 to approximately 60° C., particularly, a temperature of approximately 50 to 60° C. Within this temperature range, the active ingredients are not damaged by heat, and also have higher extraction efficiency compared to when the active ingredients are extracted at a temperature lower than the temperature range. Also, the stirring rate may be relatively low (approximately 50 to approximately 100 rpm). Within this stirring rate range, damage of the active ingredients may be reduced, and the extraction efficiency of the active ingredients may also be improved. Also, the content of the active ingredients may be remained stable.

Also, the powder of Smilax china L. root may be included at a content of approximately 1 to approximately 10 g, based on 100 ml of the aqueous solution of ethanol. Within this content range, the content of the active ingredients may increase.

Then, the ethanol extract of Smilax china L. root is filtered to obtain a filtrate (S30). The filtrate may be concentrated to obtain a concentrate having reduced ethanol content (S40). The concentration may be a concentration performed under a reduced pressure, particularly, a concentration performed under reduced-pressure and heating conditions. In this case, the concentration performed under reduced-pressure and heating conditions may be carried out at a temperature of approximately 40 to 80° C. This is a temperature range set in consideration of the boiling point of ethanol. That is, the temperature range may be adjusted to prevent the ethanol extract of Smilax china L. root from flowing backward to a concentrator pipe as the boiling point of ethanol decreases under a reduced pressure. In this case, the concentrate may have reduced ethanol content, or ethanol may be removed from the concentrate.

Such a filtrate and concentrate of the Smilax china L. root extract may include oxyresveratrol and resveratrol as active ingredients. More particularly, the Smilax china L. root extract may include resveratrol at approximately 300 to approximately 480 parts by weight, particularly at approximately 350 to approximately 430 parts by weight, and, more particularly at approximately 370 to approximately 410 parts by weight, based on 100 parts by weight of oxyresveratrol. By way of example, the Smilax china L. root extract may include oxyresveratrol at approximately 10 to 50 ppm, particularly at approximately 20 to 40 ppm, and resveratrol at approximately 100 to 200 ppm, particularly at approximately 100 to approximately 150 ppm.

Subsequently, the concentrate may be dried to prepare an extract powder, or the concentrate may be diluted with water to prepare a proper diluted solution of the extract. The drying may be freeze drying or spray drying. Meanwhile, the water may be distilled water.

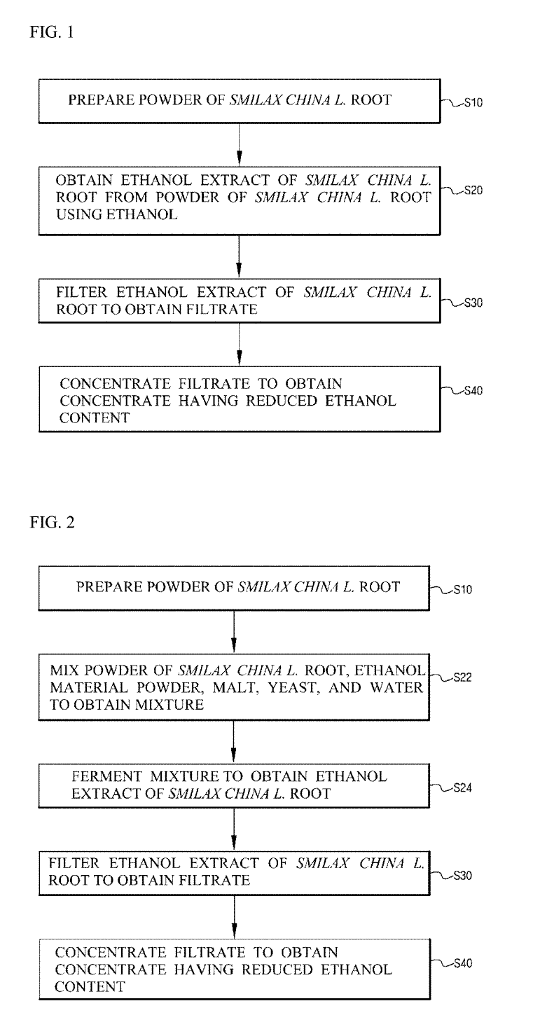

FIG. 2 is a flowchart illustrating a method for preparing a Smilax china L. root extract according to another embodiment of the present invention. The method for preparing a Smilax china L. root extract according to this embodiment may be similar to the method for preparing a Smilax china L. root extract as described with reference to FIG. 1, except the contents to be described below.

Referring to FIG. 2, a powder of Smilax china L. root is prepared (S10). In this regard, see the corresponding contents as described with reference to FIG. 1.

Next, the powder of Smilax china L. root, an ethanol material powder, a malt, a yeast, and water are mixed to obtain a mixture (S22). The water may be distilled water. The ethanol material powder may be a grain flour, particularly a kind of powder selected from the group consisting of rice flour, wheat flour, barley flour, corn flour, sorghum flour, and millet flour. The malt may be a kind of malt selected from the group consisting of Aspergillus qwamori, Aspergillus saitoi, Aspergillus usami, and Aspergillus oryzae. The yeast may be a kind of yeast selected from the group consisting of Brettanomyces, Candida, Kloeckera, Saccharomycodes, Zygosaccharomyces, Aureobasidium, and Ra parisienne (also referred to as Saccharomyces cerevisiae).

In this case, when the ethanol material powder is present at a content of 100 parts by weight in the mixture, the powder of Smilax china L. root may be included at a content of approximately 1 to approximately 10 parts by weight. Also, the malt may be included at contents of approximately 0.5 to 1.5 parts by weight, the yeast may be included at contents of approximately 0.1 to approximately 1 part by weight, and the distilled water may be included at contents of approximately 100 to 300 parts by weight.

The mixture is fermented to obtain an ethanol extract of Smilax china L. root (S24). During the fermentation of the mixture, the ethanol material powder may be glycosylated by the malt, and then fermented by the yeast to produce ethanol. The active ingredients may be extracted from the powder of Smilax china L. root due to the presence of ethanol. The fermentation may be carried out until the alcohol content reaches approximately 15 to 30%, particularly approximately 17 to 20%. Also, the fermentation may be carried out at room temperature. In this fermentation process, the content of the active ingredients, oxyresveratrol and resveratrol per gram of the powder of Smilax china L. root, may further increase, compared to that of Example as described with reference to FIG. 1.

Also, the ethanol extract of Smilax china L. root may further include piceid as an active ingredient in addition of the oxyresveratrol and resveratrol. Piceid is a glycoside of resveratrol which is a bioactive substance that is reported to have a function to prevent cardiovascular diseases and aging which becomes an issue currently. Meanwhile, such piceid may be converted into resveratrol during a fermentation process, so that the content of resveratrol per gram of the powder of Smilax china L. root in the extract can increase, compared to that of Example as described with reference to FIG. 1.

Subsequently, the ethanol extract of Smilax china L. root may be filtered to obtain a filtrate (S30). The filtrate may be concentrated to obtain a concentrate having reduced ethanol content (S40). In this regard, see the corresponding contents as described with reference to FIG. 1.

Then, the concentrate may be dried to prepare an extract powder, or the concentrate may be diluted with water to prepare a proper diluted solution of the extract. The drying may be freeze drying or spray drying. Meanwhile, the water may be distilled water.

Beverage Composition for Detoxification Including Smilax China L. Root Extract

The beverage composition for detoxification according to one embodiment of the present invention may include the Smilax china L. root extract, a weak acid, a sugar alcohol, a flavoring agent, and a solvent.

The Smilax china L. root extract may be a concentrate, a solid preparation prepared by processing the concentrate, or a diluted solution obtained by diluting the concentrate, as described with reference to FIGS. 1 and 2. The Smilax china L. root extract may have a density of approximately 10 to 25 mg/ml.

The weak acid may include at least one selected from the group consisting of citric acid, malic acid, tartaric acid, acetic acid, oxalic acid, tannic acid, butyric acid, lactic acid, glacial acetic acid, and ascorbic acid. The sugar alcohol may include at least one selected from the group consisting of xylitol, isomalt, maltitol, sorbitol, erythritol, mannitol, lactitol, and manatol. The flavoring agent may be a fruit, a fruit extract, a herb, or a herb extract. The fruit may be an apple, a lemon, an orange, a grape, a strawberry, or a peach, and the herb may be peppermint, rosemary, lavender, or lemon balm. In this case, the weak acid and the sugar alcohol may be added to improve bioabsorptivity of the active ingredients, and the flavoring agent may be added so that a human being can feel senses of flavoring, for example, refreshing or sweet fragrances and tastes when the human being eats beverages.

In the beverage composition for detoxification, the Smilax china L. root extract may be present at content of approximately 0.1 to approximately 0.25% (w/v), particularly at content of approximately 0.15 to approximately 0.21% (w/v), and more particularly at content of approximately 0.16 to approximately 0.2% (w/v); the weak acid may be present at content of approximately 0.01 to approximately 0.15% (w/v), particularly at content of approximately 0.05 to approximately 0.13% (w/v), more particularly at content of approximately 0.07 to approximately 0.10% (w/v), or approximately 0.07 to approximately 0.09% (w/v), and further particularly at content of approximately 0.08 to approximately 0.10% (w/v), or approximately 0.08 to approximately 0.09% (w/v); the sugar alcohol may be present at a content of approximately 5.5 to approximately 8% (w/v), particularly at content of approximately 5.8 to approximately 7.8% (w/v), more particularly at content of approximately 6.3 to approximately 7.1% (w/v), or approximately 6.3 to approximately 6.8% (w/v), and further particularly at content of approximately 6.5 to approximately 7.1% (w/v), or approximately 6.5 to approximately 6.8% (w/v); the flavoring agent may be present at a content of approximately 1 to 2.6% (w/v), particularly at content of approximately 1.4 to approximately 2.3% (w/v), more particularly at content of approximately 1.6 to approximately 2.0% (w/v), or approximately 1.6 to approximately 1.9% (w/v), and further particularly at content of approximately 1.7 to approximately 2.0% (w/v), or approximately 1.7 to approximately 1.9% (w/v); and the balance may be water. When the respective components are added in this ratio, the components may be admixed with the Smilax china L. root extract to exhibit effective sensory characteristics as a beverage.

More particularly, the weak acid may be either citric acid or tartaric acid that tastes sour and refreshing, is easily dissolved in water or alcohol, is stable, and has low moisture absorbing properties. In particular, the weak acid may be citric acid. The sugar alcohol may be one of maltitol and xylitol that are low in calories and 60 to 70% lower in sweetness than sugar. In particular, the sugar alcohol may be xylitol.

The flavoring agent may be a lemon concentrate or peppermint. When the flavoring agent is a lemon concentrate and peppermint, the beverage composition for detoxification may include the lemon concentrate at approximately 1 to 2.5% (w/v), particularly at approximately 1.4 to approximately 2.2% (w/v), more particularly at approximately 1.6 to approximately 1.9% (w/v), or approximately 1.6 to approximately 1.8% (w/v), and further particularly at approximately 1.7 to approximately 1.9% (w/v), or approximately 1.7 to approximately 1.8% (w/v); and the peppermint at approximately 0.001 to 0.1% (w/v), particularly at approximately 0.01 to 0.03% (w/v), more particularly at approximately 0.015 to 0.023% (w/v), or approximately 0.015 to 0.02% (w/v), and further particularly at approximately 0.017 to 0.023% (w/v), or approximately 0.017 to 0.02% (w/v).

As such, the beverage composition including the Smilax china L. root extract is suitable for being eaten as a beverage due to excellent sensory characteristics, and also exhibits an excellent ability to protect the liver from hepatotoxicity caused by nicotine or alcohol and an excellent inhibitory effect on formation of oxidatively damaged adducts.

Other Food Compositions or Pharmaceutical Compositions Including Smilax China L. Root Extract

In addition to the beverage composition according to one embodiment of the present invention, another food composition or pharmaceutical composition for detoxification may be similar to the beverage composition for detoxification, except the contents to be described below.

In addition to the beverage composition according to one embodiment of the present invention, another food composition or pharmaceutical composition for detoxification may include the Smilax china L. root extract, a weak acid, and a sugar alcohol. In addition, the composition may further include a flavoring agent. In addition to these, various kinds of excipients or additives such as a surfactant, a pigmenting material, a spice, a preservative, a stabilizer, a buffer, a suspending agent, and the like.

In this case, the weak acid, the sugar alcohol, and the flavoring agent may be included at contents of approximately 20 to 80 parts by weight, approximately 3,000 to 5,000 parts by weight, and approximately 700 to 1,300 parts by weight, respectively, based on 100 parts by weight of the Smilax china L. root extract.

Such a composition is administered orally, and may be applied to various formulations such as a tablet, a pill, a capsule, a granule, a caramel, a tea bag, and the like.

Hereinafter, the present invention will be described in detail referring to Examples and Comparative Examples. However, it should be understood that the description proposed herein is just an example for the purpose of illustrations only, not intended to limit the scope of the invention.

### Preparation Example 1 of Smilax China L. Root Extract

Smilax china L. root was collected at the district of Eumseong-gun, Chungcheongbuk-do, S. Korea, washed with distilled water, freeze-dried, ground finely, and then stored at −20° C. 500 ml of an aqueous solution of 50% alcohol (50% ethanol) was mixed with 25 g of the Smilax china L. root powder, and stirred at 70 rpm for 24 hours in an incubator whose inner temperature was set to 55° C. to obtain a Smilax china L. root extract.

The Smilax china L. root extract was filtered to obtain a filtrate (in which the concentrations of oxyresveratrol and resveratrol were 31.03 ppm and 120.48 ppm, respectively). Then, the filtrate was concentrated 10 times at 50° C. under a reduced pressure to obtain a concentrate (in which the concentrations of oxyresveratrol and resveratrol were 310.3 ppm and 1204.8 ppm, respectively). Then, the concentrate was diluted 10 times with distilled water to obtain a diluted solution having a density of 1.8 mg/ml (in which the concentrations of oxyresveratrol and resveratrol were 31.03 ppm (2.1 μM) and 120.48 ppm (0.5 μM), respectively).

### Evaluation Example 1: Separation and Identification of Functional Materials of Smilax China L. Root Extract According to Preparation Example 1

To separate functional materials in the extract (i.e., a diluted solution) obtained in Preparation Example 1 of Smilax china L. root extract, UPLC-PDA-ESI-MS/MSn (LCQ fleet, Thermo Scientific, USA) equipped with a C₁₈Hypersil GOLD column (50×2.1 mm, 1.9 μm, Thermo Scientific, USA) was used. In this case, 0.1% formic acid in water and acetonitrile were used as mobile phases A and B, the flow velocity was set to 0.2 ml/min, the concentration gradient was set to 82% A at 0 minute, 75% A at 1.96 minutes, 60% A at 5.21 minutes, 82% A at 5.5 minutes, and 82% A at 8.5 minutes, and the amount of injection was set to 1 μl. The analysis was performed at a PDA wavelength of 320 nm, the ionization was performed in an ESI positive-ion mode, and the detection was performed in a mass range of 50 to 800 (m/z) and a set capillary temperature of 275° C. All the samples were filtered through a 0.2 μm filter before the samples were injected into a UPLC analyzer, and then analyzed.

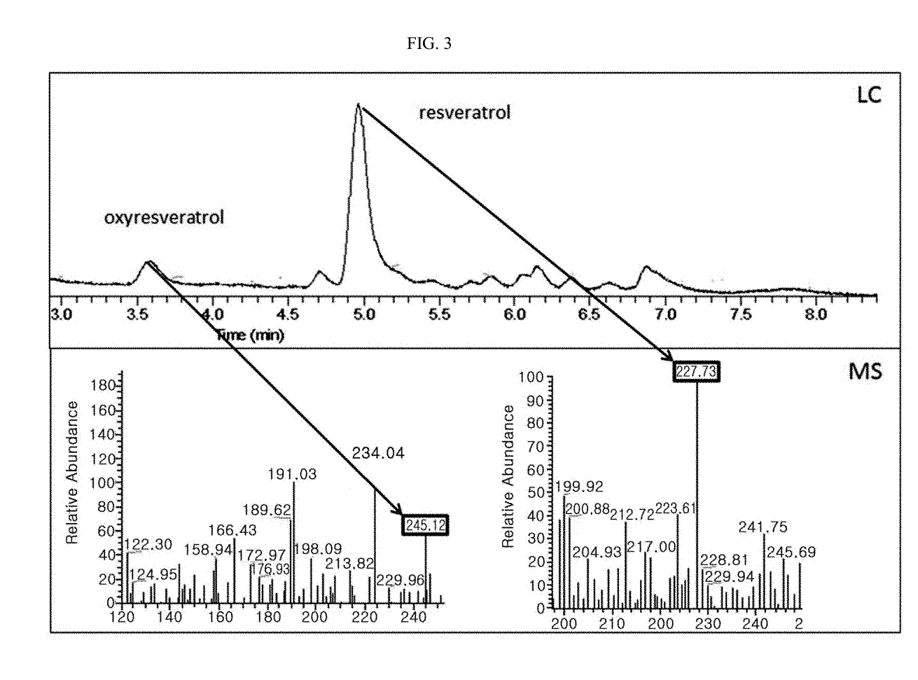

FIG. 3 is a graph showing the results of separating and identifying the functional materials of the Smilax china L. root extract according to Evaluation Example 1.

Referring to FIG. 3, major functional materials, oxyresveratrol and resveratrol, were detected at time points of 3.58 minutes and 4.9 minutes, respectively.

In the MS analysis, m/z 245.13 [M-H]⁺ and 229.02 [M-H]⁺ were observed to verify the presence of oxyresveratrol and resveratrol. Also, the contents of oxyresveratrol and resveratrol were 31.03 μg/ml and 120.48 μg/ml, respectively. That is, it was revealed that the content of resveratrol was 3.88 times that of oxyresveratrol.

### Evaluation Example 2: Evaluation of Protective Effect of Functional Index Materials in Smilax China L. Root Extract According to Preparation Example 1 on Hepatotoxicity Caused by Nicotine (MTT Assay)

A protective effect of major functional index materials, oxyresveratrol and resveratrol, in the Smilax china L. root extract on hepatotoxicity caused by nicotine was evaluated using a microculture tetrazolium assay (MTT assay). A tetrazolium-based colorimetric assay (i.e., MTT assay) is a method of dissolving water-insoluble formazan crystals, which were produced by MTT in cells and passed through cell membranes, in dimethyl sulfoxide (DMSO) and measuring an amount of the produced formazan crystals using a spectroscopic method. Here, the cell viability may be measured using this MTT assay.

HepG2 (passage #99-100) cells kindly provided from the Korean Cell Line Bank were sub-cultured, divided into a 96 well plate at a density of 1×10⁴ cells, and then cultured for 24 hours under conditions of 5% CO₂ and 37° C. After 24 hours, the HepG2 cells of experimental groups which were treated with 50, 100, and 250 μM of oxyresveratrol (commercially available from Sigma-Aldrich), 50, 100, and 250 μM of resveratrol (commercially available from Sigma-Aldrich), 50 and 100 μM of a mixture of oxyresveratrol and resveratrol (a molar ratio=1:1), and the Smilax china L. root extract according to Preparation Example 1 (a concentration of 1.8 mg/ml, including 2.1 μM of resveratrol and 0.5 μM of oxyresveratrol) were cultured for 2 hours. On the other hand, a group in which the cells were treated with neither oxyresveratrol nor resveratrol was used as the control. Thereafter, the experimental groups and the control were treated with nicotine at an IC₅₀ concentration, and cultured for 24 hours. After the medium was removed, the experimental groups and the control were treated with a 3-4,5 dimethylthiazol-2,5 diphenyltetrazolium bromide (MTT reagent), and then cultured for 4 hours. Then, the medium was removed, and the experimental groups and the control were treated with a DMSO reagent, and measured for absorbance at a wavelength of 570 nm using a multi-microplate reader (Thermo scientific). Also, the cell viability was calculated using the following Equation 1.

$$\% cell viability = \frac{\begin{matrix}Average of control ( O D ) - \\ Average of test ( O D )\end{matrix}}{\begin{matrix}Average of test ( O D ) - \\ Average of blank ( O D )\end{matrix}} * 100 \qquad [ Equation 1 ]$$

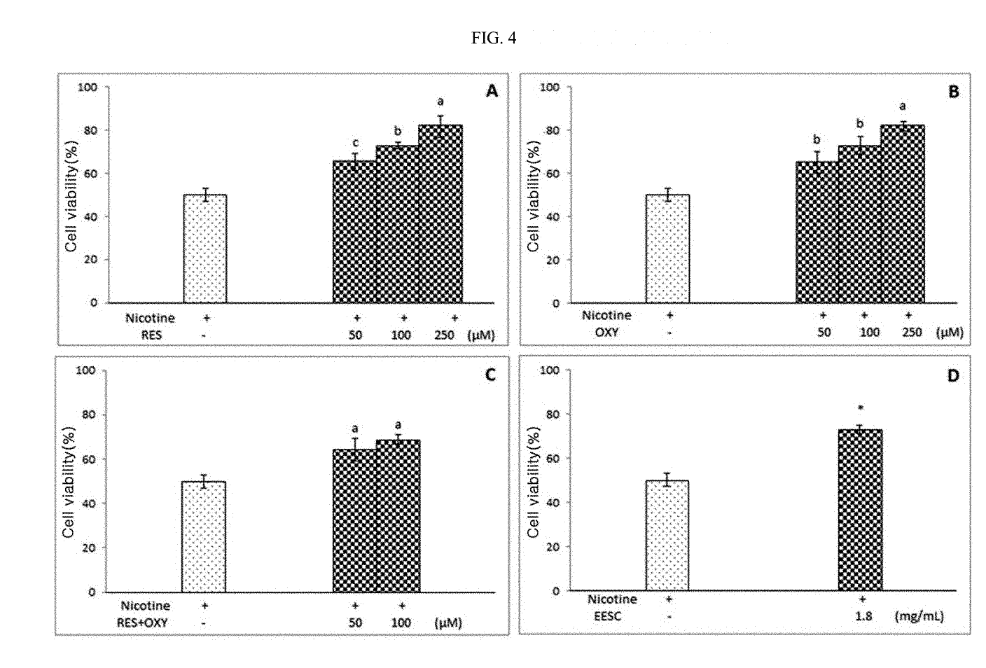

FIG. 4 is a graph showing the cell viabilities in the experimental groups and the control according to Evaluation Example 2. RES represents resveratrol, OXY represents oxyresveratrol, RES+OXY represents a mixture of resveratrol and oxyresveratrol, and EESC represents a Smilax china L. root extract. Also, “+” represents that cells are treated with the substance, and “−” represents that cells are not treated with the substance.

Referring to FIG. 4, it could be seen that the cell viability was enhanced, that is, the protective effect on cytotoxicity was improved, as the concentration of the index material increased in the experimental groups in which the cells were treated with oxyresveratrol (OXY, FIG. 4B), resveratrol (RES, FIG. 4A), and the mixture of oxyresveratrol and resveratrol (RES+OXY, FIG. 4C), compared to the control in which the cells were treated only with nicotine. Also, it could be seen that the cell viability was enhanced, that is, the protective effect on cytotoxicity was improved, in the experimental group (FIG. 4D) in which the cells were treated with the Smilax china L. root extract (EESC), compared to the control in which the cells were treated only with nicotine. Further, it was revealed that the experimental group in which the cells were treated with the Smilax china L. root extract (EESC) showed the substantially similar protective effect on cytotoxicity to the other experimental groups even when the molar concentrations of resveratrol and oxyresveratrol in the Smilax china L. root extract (EESC) were 2.1 μM and 0.5 μM, respectively, which were lower in the order of approximately 10 times or more than the other experimental groups.

### Evaluation Example 3: Evaluation of Inhibitory Effect of Functional Index Materials in Smilax China L. Root Extract According to Preparation Example 1 on Formation of Oxidatively Damaged Adducts Cuased by Nicotine (DCFH-DH Assay)

The inhibitory effect of the major functional index materials, oxyresveratrol and resveratrol, in the Smilax china L. root extract on formation of oxidatively damaged adducts caused by nicotine was evaluated using a dichlorofluorescin diacetate (DCFH-DA) assay. When non-polar, uncharged DCFH-DA easily enters the cytoplasm through cell membranes, DCFH-DA is converted into 2′,7′-dichlorofluorescin (DCFH) showing no fluorescence by an intracellular enzyme, esterase, and DCFH is then oxidized into 2,40 ,7′-dichlorofluorescein (DCF) showing fluorescence by various active oxygen produced in the cells. Since the measured fluorescence intensity is in proportion to a degree of intracellular oxidation, an effect of the index materials on oxidative damage of liver cells by nicotine was evaluated using the principle.

HepG2 (passage #99-100) cells kindly provided from the Korean Cell Line Bank were sub-cultured, divided into a 96 well plate at a density of 1×10⁴ cells, and then cultured for 24 hours under conditions of 5% CO₂ and 37° C., and the medium was removed. Then, the HepG2 cells of experimental groups which were treated with 50, 100, and 250 μM of oxyresveratrol (commercially available from Sigma-Aldrich), 50, 100, and 250 μM of resveratrol (commercially available from Sigma-Aldrich), 50 and 100 μM of a mixture of oxyresveratrol and resveratrol (a molar ratio=1:1), and the Smilax china L. root extract according to Preparation Example 1 (a concentration of 1.8 mg/ml, including 2.1 μM of resveratrol and 0.5 μm of oxyresveratrol) were cultured for 2 hours. On the other hand, a group in which the cells were treated with neither oxyresveratrol nor resveratrol was used as the control. Thereafter, the experimental groups and the control were treated with nicotine at an IC₅₀ concentration, and cultured for 24 hours. Trypsin-EDTA was divided, and the cells were detached, and treated with a 5 μM DCFH-DA reagent. After 30 minutes, the fluorescence was measured at an excitation wavelength of 488 nm and an emission wavelength of 525 nm using a multi-microplate reader (Thermo scientific).

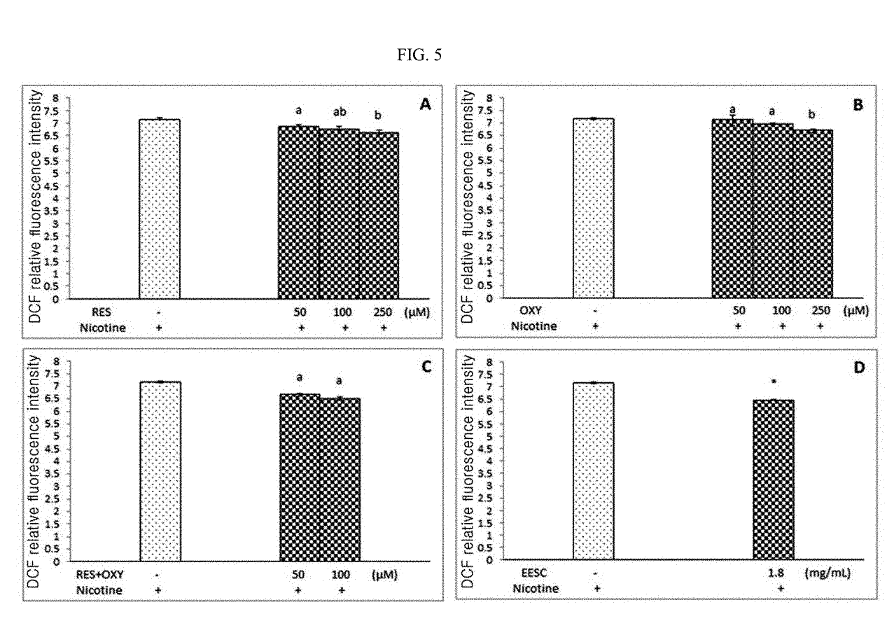

FIG. 5 is a graph showing the relative DCF fluorescence intensities in the experimental groups and the control according to Evaluation Example 3. RES represents resveratrol, OXY represents oxyresveratrol, RES+OXY represents a mixture of resveratrol and oxyresveratrol, and EESC represents a Smilax china L. root extract. Also, “+” represents that cells are treated with the substance, and “−” represents that cells are not treated with the substance.

Referring to FIG. 5, it could be seen that the relative DCF fluorescence intensity tended to decrease, that is, the formation of the oxidatively damaged adducts by nicotine tended to decrease, as the concentration of the index material increased in the experimental groups in which the cells were treated with oxyresveratrol (OXY, FIG. 5B), resveratrol (RES, FIG. 5A), and the mixture of oxyresveratrol and resveratrol (RES+OXY, FIG. 5C), compared to the control in which the cells were treated only with nicotine. Also, it could be seen that the relative DCF fluorescence intensity tended to decrease, that is, the formation of the oxidatively damaged adducts by nicotine tended to decrease, in the experimental group (FIG. 5D) in which the cells were treated with the Smilax china L. root extract (EESC), compared to the control in which the cells were treated only with nicotine. Further, it was revealed that the experimental group in which the cells were treated with the Smilax china L. root extract (EESC) showed the substantially similar inhibitory effect on the formation of oxidatively damaged adducts to the other experimental groups even when the molar concentrations of resveratrol and oxyresveratrol in the Smilax china L. root extract (EESC) were 2.1 μM and 0.5 μM, respectively, which were lower in the order of approximately 10 times or more than the other experimental groups.

### Evaluation Example 4: Evaluation of Conversion Rate of Nicotine into Cotinine by Functional Index Materials in Smilax China L. Root Extract According to Preparation Example 1 (Cotinine Quantitative Assay)

The conversion rate of nicotine into cotinine by the major functional index materials, oxyresveratrol and resveratrol, in the Smilax china L. root extract was evaluated.

HepG2 (passage #99-100) cells kindly provided from the Korean Cell Line Bank were sub-cultured, divided into a 96 well plate, and then cultured under conditions of 5% CO₂ and 37° C. until the cells reached 100% confluence, and the medium was removed. Thereafter, the HepG2 cells were cultured for a day in a medium containing 1 mM nicotine. Then, experimental groups in which the cells were treated with 50, 100, and 250 μM of oxyresveratrol (commercially available from Sigma-Aldrich), 50, 100, and 250 μM of resveratrol (commercially available from Sigma-Aldrich), 50 and 100 μM of a mixture of oxyresveratrol and resveratrol (a molar ratio=1:1), and the Smilax china L. root extract according to Preparation Example 1 (a concentration of 1.8 mg/ml, including 2.1 μM resveratrol and 0.5 μM oxyresveratrol) were divided, and a group in which the cells were treated with neither oxyresveratrol nor resveratrol was used as the control. Considering that the half-life of nicotine was 2 hours, the reactions on the experimental groups and the control were terminated at time points of 10, 20, 30, 60, 90, and 120 minutes. Subsequently, the cultured cells were dividedly treated with trypsin-EDTA, and detached, and centrifuged to collect a cell pellet. The cell pellet was suspended in 100 μl of PBS, and homogenated for 30 seconds using an ultrasonicator. 100 μl of a 4M sodium acetate buffer (pH 4.7), 40 μl of 1.5M potassium cyanide, 40 μl of 0.4M chloramine T, and 200 μl of 78 mM barbituric acid (in acetone/water (50/50)) were sequentially added to the homogenated cells, mixed for 10 seconds, and then reacted at room temperature for 15 minutes. Then, 40 μl of 1M sodium metabisulfite was added to the reaction mixture to stop the reaction. Then, the reaction mixture was measured for absorbance at a wavelength of 490 nm using a multi-microplate reader (Thermo scientific), and the absorbance of the reaction mixture was quantified by comparison to the standard curve of a cotinine sample.

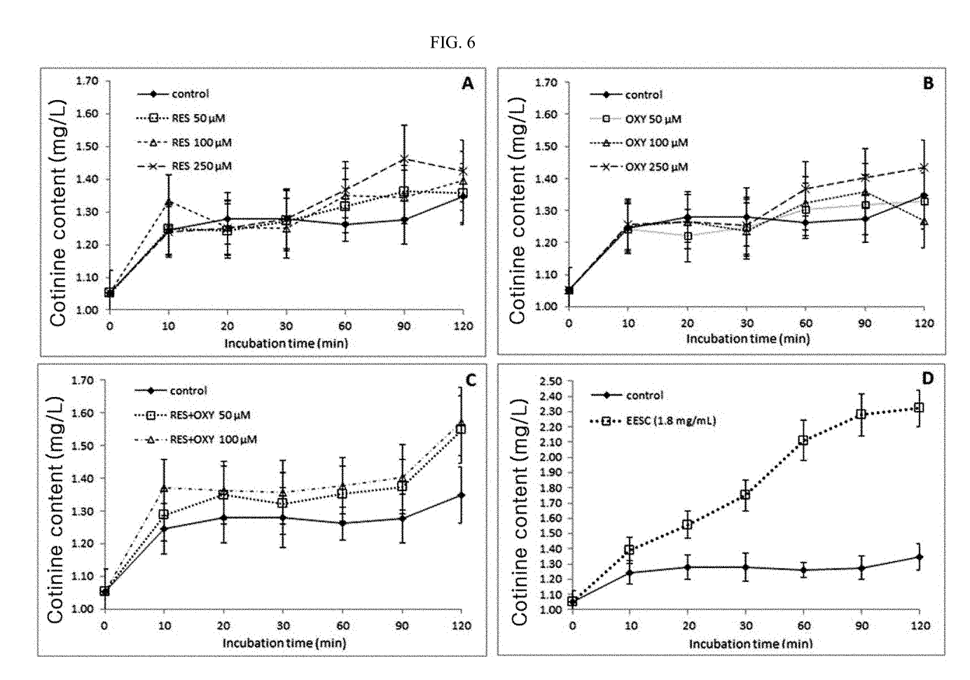

FIG. 6 is a graph obtained by plotting the cotinine contents against incubation times in the experimental groups and the control according to Evaluation Example 4. RES represents resveratrol, OXY represents oxyresveratrol, RES+OXY represents a mixture of resveratrol and oxyresveratrol, and EESC represents a Smilax china L. root extract.

Referring to FIG. 6, it could be seen that the cotinine conversion rate increased in the experimental groups in which the cells were treated with oxyresveratrol (OXY, FIG. 6B), resveratrol (RES, FIG. 6A), the mixture of oxyresveratrol and resveratrol (RES+OXY, FIG. 6C), and the Smilax china L. root extract (EESC), compared to the control in which the cells were treated only with nicotine. Further, it could be seen that the experimental group (FIG. 6D) in which the cells were treated with the Smilax china L. root extract (EESC) had the highest cotinine conversion rate. In addition, it was revealed that the experimental group in which the cells were treated with the Smilax china L. root extract (EESC) rather had a higher cotinine conversion rate even when the molar concentrations of resveratrol and oxyresveratrol in the Smilax china L. root extract (EESC) were 2.1 μM and 0.5 μM, respectively, which were lower in the order of approximately 10 times or more than the other experimental groups.

### Evaluation Example 5: Evaluation of Effect of Functional Index Materials in Smilax China L. Root Extract According to Preparation Example 1 on Expression Level of CYP2A6 Enzyme

A CYP2A6 enzyme is associated with conversion of nicotine into cotinine in the liver, and thus the expression level of the CYP2A6 enzyme may be used to evaluate how the functional index materials play a part in conversion into cotinine.

HepG2 (passage #99-100) cells kindly provided from the Korean Cell Line Bank were sub-cultured, divided into a 96 well plate at a density of 1×10⁵ cells, and then cultured for 24 hours under conditions of 5% CO₂ and 37° C., and the medium was removed. The HepG2 cells of the experimental groups which were pretreated with 250 μM of resveratrol (commercially available from Sigma-Aldrich), 250 μM of oxyresveratrol (commercially available from Sigma-Aldrich), 100 μM of the mixture of oxyresveratrol and resveratrol (a molar ratio=1:1), and the Smilax china L. root extract according to Preparation Example 1 (a concentration of 1.8 mg/ml, including 2.1 μM of resveratrol and 0.5 μM of oxyresveratrol), all of which showed high effects in the evaluation using the cotinine quantitative assay according to Evaluation Example 4, were cultured for 120 minutes. On the other hand, a group in which the cells were treated with neither oxyresveratrol nor resveratrol was used as the control. Thereafter, the cultured cells were dividedly treated with trypsin-EDTA, and detached, and centrifuged at 1,500 g for 5 minutes, and the supernatant was discarded. Total RNA was extracted from the cell pellet obtained through the centrifugation using Trizol, and cDNA complementary to the RNA was synthesized using an iScript cDNA synthesis kit. The synthesized cDNA was analyzed to evaluate an expression level of a CYP2A6 enzyme using Stratagene MX3005P.

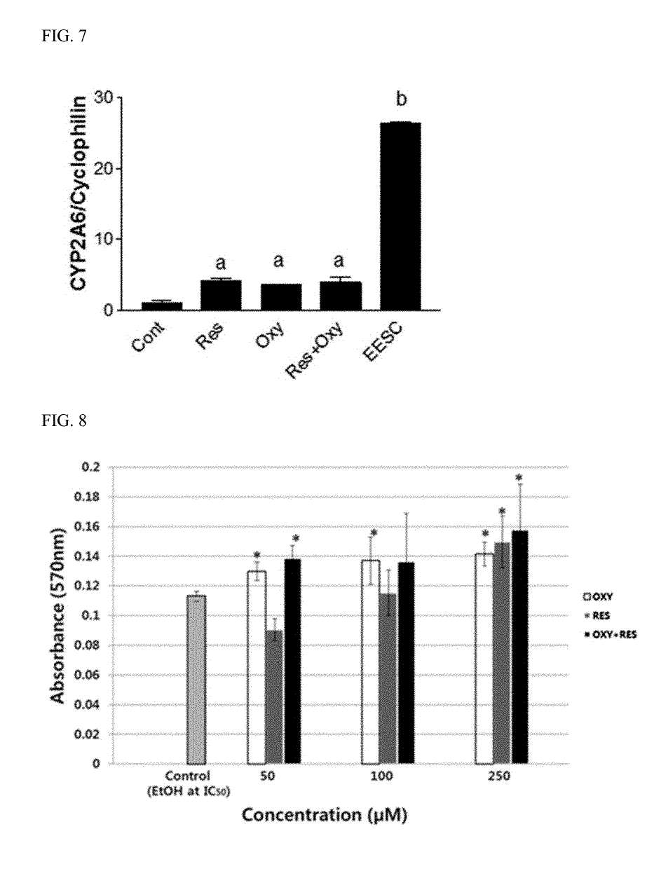

FIG. 7 is a graph illustrating the expression levels of the CYP2A6 enzyme in the experimental groups and the control according to Evaluation Example 5. RES represents resveratrol, OXY represents oxyresveratrol, RES+OXY represents a mixture of resveratrol and oxyresveratrol, and EESC represents a Smilax china L. root extract.

Referring to FIG. 7, it could be seen that the enzyme expression levels increased in all the experimental groups in which the cells were treated with oxyresveratrol (OXY), resveratrol (RES), the mixture of oxyresveratrol and resveratrol (RES+OXY), and the Smilax china L. root extract (EESC), compared to the control (Cont) in which the cells were treated only with nicotine. More particularly, it was shown that the expression levels of the CYP2A6 enzyme increased by at least 2.9 times up to 10.4 times. In particular, it was revealed that the experimental group in which the cells were treated with the Smilax china L. root extract (EESC) had the highest enzyme expression level even when the molar concentrations of resveratrol and oxyresveratrol in the Smilax china L. root extract (EESC) were 2.1 μM and 0.5 μM, respectively, which were lower in the order of approximately 10 times or more than the other experimental groups, indicating that a synergistic effect was exerted due to the presence of various bioactive substances in addition to oxyresveratrol and resveratrol in the Smilax china L. root extract (EESC). From these facts, it could be seen that the Smilax china L. root extract had an important role in the metabolism of nicotine into cotinine by inducing expression of the CYP2A6 enzyme in the liver.

### Evaluation Example 6: Evaluation of Protective Effect of Functional Index Materials in Smilax China L. Root Extract According to Preparation Example 1 on Hepatotoxicity Caused by Alcohol (MTT Assay)

A protective effect of the major functional index materials, oxyresveratrol and resveratrol, in the Smilax china L. root extract on hepatotoxicity caused by alcohol was evaluated using an MTT assay. The MTT assay was generally described with reference to Evaluation Example 2.

HepG2 (passage #16-20) cells kindly provided from the Korean Cell Line Bank were sub-cultured, divided into a 96 well plate at a density of 1×10⁴ cells, and then cultured for 24 hours under conditions of 5% CO₂ and 37° C. After 24 hours, the HepG2 cells of experimental groups which were treated with 50, 100, and 250 μM of oxyresveratrol (commercially available from Sigma-Aldrich), 50, 100, and 250 μM of resveratrol (commercially available from Sigma-Aldrich), and 50, 100, and 250 μM of a mixture of oxyresveratrol and resveratrol (a molar ratio=1:1) were cultured for 2 hours. In this case, a group in which the cells were treated with neither oxyresveratrol nor resveratrol was used as the control. Thereafter, the experimental groups and the control were treated with an aqueous solution of ethanol (50%) at an IC₅₀ concentration, and cultured for 24 hours. After the medium was removed, the experimental groups and the control were treated with a 3-4,5 dimethylthiazol-2,5 diphenyltetrazolium bromide (MTT) reagent, and then cultured for 4 hours. Then, the medium was removed, and the experimental groups and the control were treated with a DMSO reagent, and measured for absorbance at a wavelength of 570 nm using a multi-microplate reader (Thermo scientific). Also, the cell viability was calculated using Equation 1.

FIG. 8 is a graph illustrating the absorbance according to the concentrations in the experimental groups and the control according to Evaluation Example 6. RES represents resveratrol, OXY represents oxyresveratrol, and RES+OXY represents a mixture of resveratrol and oxyresveratrol.

Referring to FIG. 8, it could be seen that the absorbance increased as the concentration of the index material increased in the experimental groups in which the cells were treated with oxyresveratrol (OXY), resveratrol (RES), and the mixture of oxyresveratrol and resveratrol (RES+OXY), compared to the control in which the cells were treated only with alcohol.

### Evaluation Example 7: Evaluation of Inhibitory Effect of Functional Index Materials in Smilax China L. Root Extract According to Preparation Example 1 on Formation of Oxidatively Damaged Adducts Caused by Alcohol (DCFH-DA Assay)

The inhibitory effect of the major functional index materials, oxyresveratrol and resveratrol, in the Smilax china L. root extract on formation of oxidatively damaged adducts caused by alcohol was evaluated using a dichlorofluorescein diacetate (DCFH-DA) assay. The DCFH-DA assay was generally described with reference to Evaluation Example 3.

HepG2 (passage #16-20) cells kindly provided from the Korean Cell Line Bank were sub-cultured, divided into a 96 well plate at a density of 1×10⁵ cells, and then cultured for 24 hours under conditions of 5% CO₂ and 37° C., and the medium was removed. Then, the HepG2 cells of experimental groups which were treated with 50, 100, and 250 μM of oxyresveratrol (commercially available from Sigma-Aldrich), 50, 100, and 250 μM of resveratrol (commercially available from Sigma-Aldrich), and 50, 100, and 250 μM of a mixture of oxyresveratrol and resveratrol (a molar ratio=1:1) were cultured for 2 hours. In this case, a group in which the cells were treated with neither oxyresveratrol nor resveratrol was used as the control. Thereafter, the experimental groups and the control were treated with ethanol at an IC₅₀ concentration, and cultured for 24 hours. Then, trypsin-EDTA was divided, and the cells were detached, and treated with a 5 μM DCFH-DA reagent. After 30 minutes, the fluorescence was measured at an excitation wavelength of 488 nm and an emission wavelength of 525 nm using a multi-microplate reader (Thermo scientific).

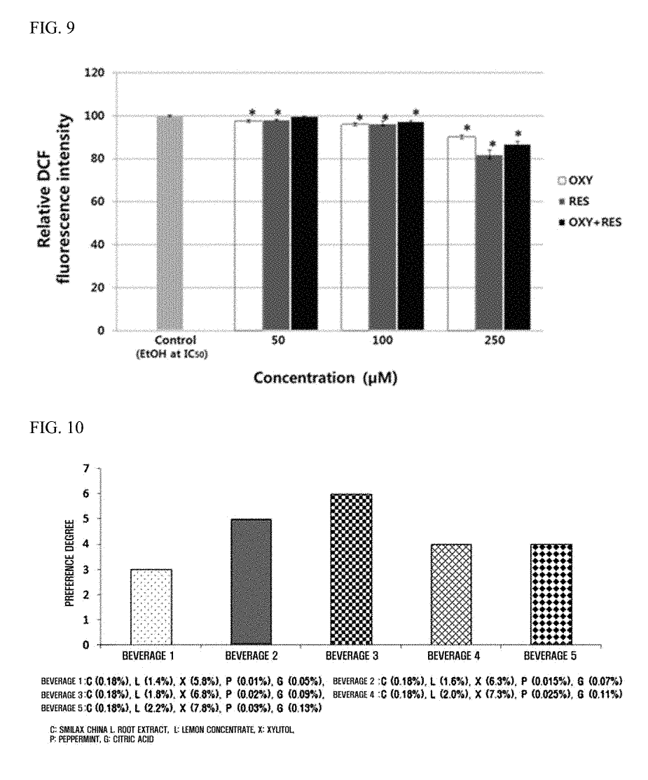

FIG. 9 is a graph illustrating the relative DCF fluorescence intensities in the experimental groups and the control according to Evaluation Example 7. RES represents resveratrol, OXY represents oxyresveratrol, and RES+OXY represents a mixture of resveratrol and oxyresveratrol.

Referring to FIG. 9, it could be seen that the relative DCF fluorescence intensity tended to decrease, that is, the formation of the oxidatively damaged adducts by alcohol tended to decrease, in the experimental groups in which the cells were treated with oxyresveratrol (OXY), resveratrol (RES), and the mixture of oxyresveratrol and resveratrol (RES+OXY), compared to the control in which the cells were treated only with alcohol.

### Preparation Example 1 of Beverage Composition Including Smilax China L. Root Extract

The extract (i.e., a concentrate) obtained in Preparation Example 1 of Smilax china L.

root extract (a density of 1.8 mg/ml, including 310.3 ppm of oxyresveratrol and 1204.8 ppm of resveratrol), a 6-Brix lemon concentrate, xylitol, peppermint, and citric acid were mixed at contents as listed in the following Table 1, and the balance of water was added to prepare 100 ml of a beverage composition including the Smilax china L. root extract. The beverage composition according to one embodiment of the present invention prepared thus included oxyresveratrol and resveratrol at final concentrations of 31.03 ppm and 120.48 ppm, respectively.

TABLE 1

| | | Smilax china L. | | | | |
|---|---|---|---|---|---|---|
| | | root extract | Lemon | | | |
| | | (Conc.: 18 mg/ml) | concentrate | Xylitol | Peppermint | Citric acid |
| Beverage 1 | Mass content | 0.18 g | 1.4 g | 5.8 g | 0.01 g | 0.05 g |
| | Part by weight | 100 | 778 | 3,222 | 5.56 | 27.78 |
| | Conc. in beverage | 0.18% | 1.4% | 5.8% | 0.01% | 0.05% |
| | (wt/v) | | | | | |
| Beverage 2 | Mass content | 0.18 g | 1.6 g | 6.3 g | 0.015 g | 0.07 g |
| | Part by weight | 100 | 889 | 3,500 | 8.33 | 38.89 |
| | Conc. in beverage | 0.18% | 1.6% | 6.3% | 0.015% | 0.07% |
| | (wt/v) | | | | | |
| Beverage 3 | Mass content | 0.18 g | 1.8 g | 6.8 g | 0.02 g | 0.09 g |
| | Part by weight | 100 | 1,000 | 3,778 | 11.11 | 50 |
| | Conc. in beverage | 0.18% | 1.8% | 6.8% | 0.02% | 0.09% |
| | (wt/v) | | | | | |
| Beverage 4 | Mass content | 0.18 g | 2.0 g | 7.3 g | 0.025 g | 0.11 g |
| | Part by weight | 100 | 1,111 | 4,056 | 13.89 | 61.11 |
| | Conc. in beverage | 0.18% | 2.0% | 7.3% | 0.025% | 0.11% |
| | (wt/v) | | | | | |
| Beverage 5 | Mass content | 0.18 g | 2.2 g | 7.8 g | 0.03 g | 0.13 g |
| | Part by weight | 100 | 1,222 | 4,333 | 16.67 | 72.22 |
| | Conc. in beverage | 0.18% | 2.2% | 7.8% | 0.03% | 0.13% |
| | (wt/v) | | | | | |

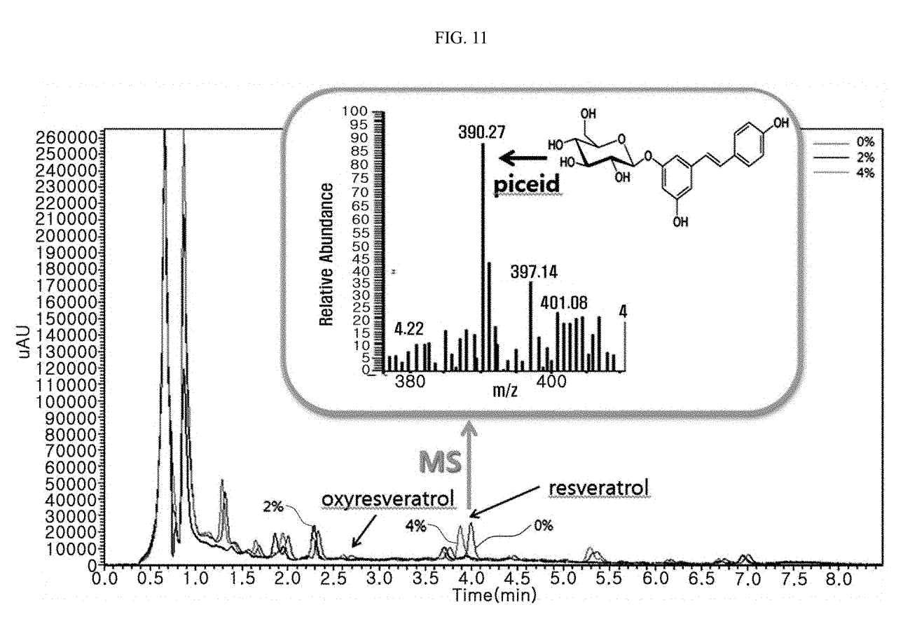

FIG. 10 is a graph showing the sensory degrees of beverages according to Preparation Example 1 of beverage composition including the Smilax china L. root extract.

The sensory degree was evaluated by providing 25 trained sensory evaluation participants with beverages 1 to 5 according to Preparation Example 1 of beverage composition, evaluating feelings of sweetness, freshness, sourness, refreshness, and bitterness using a seven-point scaling system in which the sensory feelings were ranked from “very bad (Point 1)” to “very good (Point 7).” Then, an average point for each beverage was calculated.

Referring to FIG. 10 and Table 1, it could be seen that the beverages 1 to 5 according to Preparation Example 1 of beverage composition including the Smilax china L. root extract showed good sensory degrees greater than or equal to Preference degree 3. From these facts, it could be seen that the beverage composition had good sensory degrees when the Smilax china L. root extract was added at a concentration of approximately 0.18% (w/v) to the beverage composition including citric acid at approximately 0.05 to approximately 0.13% (w/v), xylitol at approximately 5.8 to approximately 7.8% (w/v), a lemon concentrate at approximately 1.4 to approximately 2.2% (w/v), peppermint at approximately 0.01 to 0.03% (w/v), and the balance of water.

In addition, the beverages 2 and 3 had superior sensory degrees to the other beverages. From these results, it could be seen that the beverage composition showed further improved sensory degrees when the Smilax china L. root extract was added at a concentration of approximately 0.18% (w/v) to the beverage composition including citric acid at approximately 0.07 to approximately 0.09% (w/v), xylitol at approximately 6.3 to approximately 6.8% (w/v), a lemon concentrate at approximately 1.6 to approximately 1.8% (w/v), peppermint at approximately 0.015 to 0.02% (w/v), and the balance of water.

In particular, the beverage 3 showed a further improved sensory degree. From these results, it could be seen that the beverage composition showed further improved sensory degrees when the Smilax china L. root extract was added at a concentration of approximately 0.18% (w/v) to the beverage composition including citric acid at approximately 0.08 to approximately 0.09% (w/v), xylitol at approximately 6.5 to approximately 6.8% (w/v), a lemon concentrate at approximately 1.7 to approximately 1.8% (w/v), peppermint at approximately 0.017 to 0.02% (w/v), and the balance of water.

Further, the capacity and cost of the products which the participants preferred were surveyed, and the results showed that the preferred capacity and cost of the products were 350 ml and 1,200 Won, respectively.

### Preparation Example 2 of Smilax China L. Root Extract: Using 2 Parts by Weight of Powder of Smilax China L. Root Based on 100 Parts by Weight of Ground Rice

A Smilax china L. root was collected at the district of Eumseong-gun, Chungcheongbuk-do, S. Korea, washed with distilled water, freeze-dried, ground finely, and then stored at −20° C. A mixture including the powder of Smilax china L. root at 2 parts by weight based on 100 parts by weight of the ground rice was prepared by mixing 6 g of the powder of Smilax china L. root, 300 g of ground rice, 2.5 g of Aspergillus usami, 1 g of Ra parisienne, and 600 ml of distilled water. The mixture was fermented at 24° C. for 9 days until the alcohol content reached 18%. During this fermentation procedure, the extracts were collected at time points of Days 1, 3, 5, 7, and 9.

### Preparation Example 3 of Smilax China L. Root Extract: Using 4 Parts by Weight of Powder of Smilax China L. Root Based on 100 Parts by Weight of Ground Rice

An extract was prepared and collected in the same manner as in Preparation Example 2 of Smilax china L. root extract, except that a mixture including the powder of Smilax china L. root at 4 parts by weight based on 100 parts by weight of the ground rice was prepared by mixing 12 g of the powder of Smilax china L. root, 300 g of ground rice, 2.5 g of Aspergillus usami, 1 g of Ra parisienne, and 600 ml of distilled water.

### Comparative Example

An extract was prepared and collected in the same manner as in Preparation Example 2 of Smilax china L. root extract, except that the powder of Smilax china L. root was not added.

### Evaluation Example 8: Separation and Identification of Functional Materials of Smilax China L. Root Extract According to Preparation Example 22

The functional materials in the Smilax china L. root extract were separated by analyzing the Smilax china L. root extract (taken on Day 1) obtained in Preparation Example 2 of Smilax china L. root extract under the same analytical conditions as in Evaluation Example 1.

FIG. 11 is a graph showing the results of separating and identifying functional materials of the Smilax china L. root extract according to Evaluation Example 8.

Referring to FIG. 11, the major functional index materials, oxyresveratrol and resveratrol, were separated at retention times of 2.72 minutes and 3.96 minutes, respectively, on Day 1 of fermentation. In the MS analysis, m/z 243.24 EM-Hf observed at a retention time of 2.72 minutes was verified as oxyresveratrol, and 227.24 EM-Hf and 390.27 EM-Hf were detected at a retention time of 3.96 minutes, and verified as resveratrol and piceid, respectively.

### Evaluation Example 9: Separation and Identification of Functional Materials of Smilax China L. Root Extract According to Preparation Example 22

The functional materials in the Smilax china L. root extract were separated by analyzing the Smilax china L. root extract (taken on Day 5) obtained in Preparation Example 2 of Smilax china L. root extract under the same analytical conditions as in Evaluation Example 1.

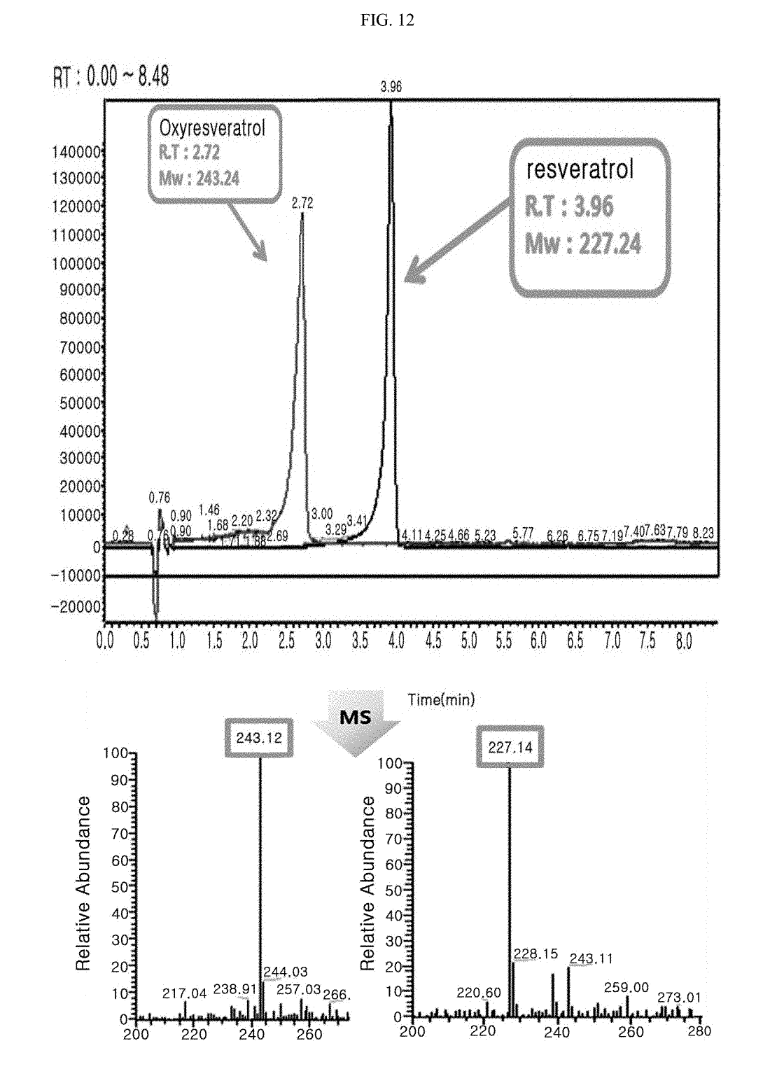

FIG. 12 is a graph showing the results of separating and identifying functional materials of the Smilax china L. root extract according to Evaluation Example 9.

Referring to FIG. 12, the major functional index materials, oxyresveratrol and resveratrol, in the Smilax china L. root extract were separated at retention times of 3.6 minutes and 5.0 minutes, respectively, on Day 5 of fermentation. In the MS analysis, m/z 245.07 [M-H]⁺ and 229.44 [M-H]⁺ were verified as oxyresveratrol and resveratrol. However, piceid was not detected. From these results, it could be assumed that piceid detected at the early stage (see FIG. 11) was gradually converted into saccharide-deficient resveratrol by means of a glycosylation action during fermentation.

### Evaluation Example 10: Analysis of Change in Contents of Resveratrol and Oxyresveratrol in Extracts According to Preparation Examples 2 and 3 and Comparative Example

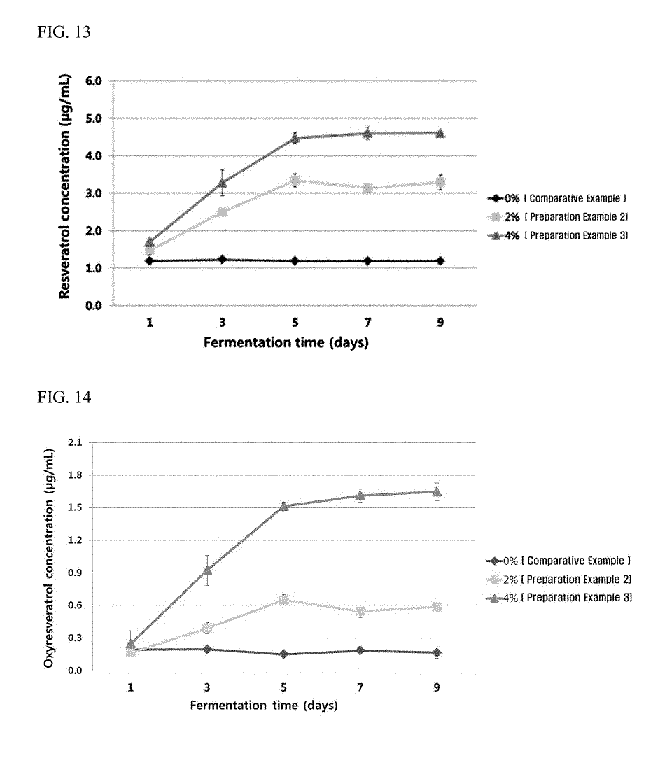

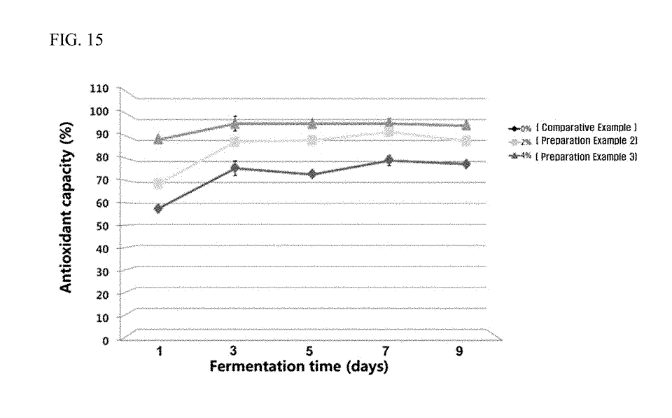

Fractions were collected from the extracts according to Preparation Examples 2 and 3 and Comparative Example on Days 1, 3, 5, 7, and 9, and the contents of resveratrol and oxyresveratrol in the fractions were analyzed through the analysis of Evaluation Example 8. The results are shown in FIGS. 13 and 14.

FIGS. 13 and 14 are graphs plotted by analyzing the changes in contents of resveratrol and oxyresveratrol in fractions collected from the extracts of Preparation Examples 2 and 3 and Comparative Example on Days 1, 3, 5, 7, and 9.

Referring to FIGS. 13 and 14, the contents of the functional materials, resveratrol and oxyresveratrol, increased between Days 1 and 5 of fermentation. More particularly, the contents of oxyresveratrol in the extracts according to Preparation Example 2 (the powder of Smilax china L. root was used at a concentration of 2% based on the total amount of the ground rice: represented by 2%) and Preparation Example 3 (the powder of Smilax china L. root was used at a concentration of 4% based on the total amount of the ground rice: represented by 4%) between Days 1 and 5 increased by 3.98 times and 6.16 times, respectively. Also, the contents of resveratrol in the extracts according to Preparation Examples 2 and 3 between Days 1 and 5 increased by 2.28 times and 2.64 times, respectively. However, it was shown that there was no significant difference in the change in the contents of resveratrol and oxyresveratrol after Day 5. This was assumed to result from that piceid detected at the early stage (see FIG. 11) was gradually converted into resveratrol by means of a glycosylation action during fermentation before Day 5 in addition to the fact that resveratrol and oxyresveratrol were increasingly extracted as the fermentation was carried out.

The contents of oxyresveratrol in the extracts collected on Day 9 of fermentation in Preparation Examples 2 and 3 were 0.59 μg/mL and 1.65 μg/mL, respectively (FIG. 14). Also, the contents of resveratrol in the extracts collected on Day 9 of fermentation in Preparation Examples 2 and 3 were 3.29 μg/mL and 4.6 μg/mL, respectively (FIG. 13). In the case of the extract according to Preparation Example 3, the contents of oxyresveratrol and resveratrol were 1.5 to 2.95 times and 1.16 to 1.47 times higher than the extract according to Preparation Example 2, respectively. From these results, it could be seen that ethanol produced by glycosylation and fermentation of rice starch using the malt (Aspergillus usami) and the yeast (Ra parisienne) served to effectively extract the major bioactive substances in the Smilax china L. root.

### Evaluation Example 11: Evaluation of Free Radical Scavenging Activity of Extracts According to Preparation Examples 2 and 3 and Comparative Example (ABTS Assay)

The free radical scavenging activity was evaluated using a 2,2′-azino-bis(3-ethylbenzothiazoline-6-sulphonic acid (ABTS) assay.

0.1 mM 2,2-azo-bis(2-methylpropion amidine)dihydrochloride (AAPH) and 2.5 mM ABTS were dissolved in a 100 mM phosphate buffer solution (PBS), and then reacted at 70° C. for 60 minutes to form ABTS free radicals. Thereafter, the resulting reaction mixture was slowly cooled at room temperature. Then, the ABTS free radical solution filtered through a 0.2 μm filter was diluted with PBS until the absorbance at 734 nm reached 0.65±0.02. 980 μl of the ABTS free radical solution, and 20 μl of each of the Smilax china L. root extracts according to Preparation Examples 2 and 3 collected on Days 1, 3, 5, 7, and 9 of fermentation were mixed, reacted at 37° C. for 10 minutes, and then measured for absorbance at 734 nm using a multi-microplate reader (commercially available from Thermo Scientific). Subsequently, a reaction solution obtained by allowing each partial sample (20 μl) of a antioxidant, vitamin C, instead of the Smilax china L. root extracts to react in 980 μl of the ABTS free radical solution was used as the control.

FIG. 15 is a graph plotted by analyzing the antioxidant capacities in the fractions collected from the extracts according to Preparation Examples 2 and 3 and Comparative Example on Days 1, 3, 5, 7, and 9.

Referring to FIG. 15, it was revealed that the antioxidant capacities did not increase after Day 3 of fermentation. Also, it was revealed that the total antioxidant capacities were improved as the content of the Smilax china L. root increased, which indicated that the antioxidant capacities were improved as the contents of the effective materials of the Smilax china L. root increased during a fermentation period.

As described above, according to the embodiments of the present invention, the Smilax china L. root extract including a large amount of oxyresveratrol and resveratrol as the active ingredients can be prepared. In addition, the ethanol extract of Smilax china L. root can further include piceid as the active ingredient in addition to oxyresveratrol and resveratrol.

Also, the beverage composition including the Smilax china L. root extract includes the active ingredients, and thus can exhibit a good ability to protect the liver from hepatotoxicity caused by nicotine or alcohol and a good inhibitory effect on formation of oxidatively damaged adducts. Further, the beverage composition exhibits excellent sensory characteristics, and thus can be suitable for being eaten as a beverage.

It will be apparent to those skilled in the art that various modifications can be made to the above-described embodiments of the present invention without departing from the scope of the invention. Thus, it is intended that the present invention covers all such modifications provided they come within the scope of the appended claims and their equivalents.

What is claimed is:

1. A method of increasing conversion rate of nicotine into cotinine in a subject, comprising administering to the subject a food including an ethanol extract of Smilax china L. root as an active ingredient.

2. The method of claim 1, wherein the expression level of the CYP2A6 enzyme in the subject increases.

3. The method of claim 1, wherein the ethanol extract of Smilax china L. root comprises oxyresveratrol and resveratrol.

4. The method of claim 3, wherein the ethanol extract of Smilax china L. root further comprises piceid.

5. The method of claim 1, wherein the food is a beverage composition, and the beverage composition comprises 0.1 to 0.25% (w/v) of the ethanol extract of Smilax china L. root, 0.01 to 0.15% (w/v) of a weak acid, 5.5 to 8% (w/v) of a sugar alcohol, 1 to 2.6% (w/v) of a flavoring agent, and the balance of water.

6. The method of claim 5, wherein the weak acid comprises at least one weak acid selected from the group consisting of citric acid, malic acid, tartaric acid, acetic acid, oxalic acid, tannic acid, butyric acid, lactic acid, glacial acetic acid, and ascorbic acid.

7. The method of claim 5, wherein the sugar alcohol comprises at least one sugar alcohol selected from the group consisting of xylitol, isomalt, maltitol, sorbitol, erythritol, mannitol, lactitol, and manatol.

8. The method of claim 5, wherein the flavoring agent comprises at least one flavoring agent selected from the group consisting of a fruit extract and a herb extract.

9. The method of claim 5, wherein the beverage composition comprises 0.15 to 0.21% (w/v) of the ethanol extract of Smilax china L. root, 0.05 to 0.13% (w/v) of the weak acid, 5.8 to 7.8% (w/v) of the sugar alcohol, 1.4 to 2.3% (w/v) of the flavoring agent, and the balance of water.

10. The method of claim 5, wherein the beverage composition comprises 0.16 to 0.2% (w/v) of the ethanol extract of Smilax china L. root, 0.07 to 0.09% (w/v) of the weak acid, 6.3 to 6.8% (w/v) of the sugar alcohol, 1.6 to 1.9% (w/v) of the flavoring agent, and the balance of water.

11. The method of claim 1, the ethanol extract of Smilax china L. root is prepared by a method comprising:
preparing a powder of a Smilax china L. root;
obtaining an ethanol extract of Smilax china L. root from the powder of Smilax china L. root using ethanol; and
concentrating the ethanol extract of Smilax china L. root under a reduced vacuum to obtain a concentrate having a reduced ethanol content.

12. The method of claim 11, wherein the obtaining of the ethanol extract of Smilax china L. root comprises:
mixing an aqueous solution of 40% to 60% ethanol with the powder of Smilax china L. root; and
stirring the resulting mixture at a temperature of 40 to 60° C. at a stirring rate of 50 to 100 rpm.

13. The method of claim 11, wherein the ethanol extract of Smilax china L. root is concentrated at a temperature of 40 to 80° C. under a reduced vacuum.

14. The method of claim 11, wherein the obtaining of the ethanol extract of Smilax china L. root comprises:
mixing the powder of Smilax china L. root, an ethanol material powder, a malt, a yeast, and water to obtain a mixture; and
fermenting the mixture to obtain an ethanol extract of Smilax china L. root.

15. The method of claim 14, wherein the ethanol material powder is at least one kind of grain flour selected from the group consisting of rice flour, wheat flour, barley flour, corn flour, sorghum flour, and millet flour.

16. The method of claim 14, wherein the malt is at least one kind of malt selected from the group consisting of Aspergillus qwamori, Aspergillus saitoi, Aspergillus usami, and Aspergillus oryzae.

17. The method of claim 14, wherein the yeast is at least one kind of yeast selected from the group consisting of Brettanomyces, Candida, Kloeckera, Saccharomycodes, Zygosaccharomyces, Aureobasidium, and Ra parisienne.

18. The method of claim 14, wherein the mixture comprises the powder of Smilax china L. root at 1 to 10 parts by weight, the malt at 0.5 to 1.5 parts by weight, the yeast at 0.1 to 1 part by weight, and the water at 100 to 300 parts by weight, based on 100 parts by weight of the ethanol material powder.

19. The method of claim 14, wherein the fermentation is carried out at room temperature until the alcohol content reaches approximately 15 to 30%.

20. The method of claim 14, wherein the ethanol extract of Smilax china L. root comprises oxyresveratrol, resveratrol, and piceid as active ingredients.
